(19) United States
(12) Patent Application Publication
(10) Pub. No.: US 2010/0274088 A1
(43) Pub. Date: Oct. 28, 2010

(54) Flexible Medical Instrument

(76) Inventors: Carl Frederic West, Waltham, MA (US); Darrell James Hartwick, Newton, MA (US)

Correspondence Address: CLOCK TOWER LAW GROUP, 2 CLOCK TOWER PLACE, SUITE 255, MAYNARD, MA, 01754-2545, US

(21) Appl. No.: 12/766,504

(22) Filed: Apr. 23, 2010

Related U.S. Application Data
(60) Provisional application No. 61/171,846, filed on Apr. 23, 2009.

(51) Int. Cl. A61B 1/00

(52) U.S. Cl. 600/140

(57) ABSTRACT

A medical device is provided for use in surgery or endoscopy. The device has a control handle, shaft, and end effector. The end effector includes a flexible bending section utilizing layered flat springs to enable two-way deflection of the bending section. Alignment, spacing, and material composition allows movement of the bending section consistent in direction and degree. Multiple bending sections may be joined sequentially to allow for multiple axis for deflection.

### CROSS-REFERENCE TO RELATED APPLICATIONS

This utility patent application claims priority from U.S. provisional patent application Ser. No. 61/171,846, filed Apr. 23, 2009, titled “Flexible medical instrument and method of manufacture” in the name of Darrell Hartwick and Carl West, which is hereby fully incorporated by reference.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. Copyright 2010, Endo-Lamina LLC.

### BACKGROUND

1. Field of Technology

This disclosure relates to medical devices. More specifically, this disclosure relates to medical devices having a flexible end and a control for the flexible end.

2. Background

Surgical and endoscopic procedures are undergoing convergence. Physicians in these and emerging specialities need improved mechanical tools. Endoscopists are increasingly capable of interventions directed at diseases including obesity and gastrointestinal cancer, but are limited by training and the availability of simple, cost-effective tools. Cardiothoracic and abdominal surgeons also demand tools with greater functionality, working through fewer and/or smaller sites (ports), or in the case of natural orifice translumenal endoscopic surgery (NOTES™), no ports at all. Reduction or elimination of external incisions provides for reduced risk of infection, reduced need for analgesia, and more rapid healing and lower overall health care costs.

Surgical and laparoscopic devices typically involve larger instruments capable of exerting reasonably high operating forces. Such instruments are often rigid to enable sufficient force, which may limit use. Endoscopic devices are typically smaller instruments to fit through working channels of existing endoscopes, often highly flexible but more limited in operating force. In both cases, applicable devices typically involve an operating portion, or end effector, connected to middle portion or shaft which in turn connects to a handle for controlling the end effector. Prior development in the art, including U.S. Pat. No. 7,670,351 (Mar. 2, 2010) by Darrell Hartwick for a for a Medical Device Using Beam Construction And Methods, have focused on the shaft and handle of the medical devices.

### BRIEF SUMMARY

Surgical and endoscopic procedures utilize medical devices having a control handle, shaft, and end effector where the control handle may direct the end effector and operate a specific tool or other instrument within the medical device. Flexibility at the end effector may be desired to direct the tool or instrument or direct movement while inserting the device. A preferred embodiment of a flexible end effector includes a flexible section having layered flat springs aligned length-wise in the direction of the shaft axis. Such layering allows flexing along a single axis while maintaining rigidity in other directions, thereby enabling two-way deflection of the flexible end effector. Layers may be connected at one end, and include lubricants to reduce sliding friction. Flat springs may be manufactured as attached with etchings to enable folding into desired alignment or other features for connection and configuration. Multiple flexible regions may be sequenced at various rotation to enable multiple axis for deflection if more than two-way deflection is required.

### BRIEF DESCRIPTION OF THE DRAWINGS

In the drawings, closely related figures and items have the same number but different alphabetic suffixes. Processes, states, statuses, and databases are named for their respective functions.

FIG. 1 is a lengthwise view of the flexible end effector connected to a shaft.

FIG. 2 is a view of the full device including handle, shaft, and end effector.

FIG. 3 is an view of an alternate embodiment of the device with flexible sections added to the shaft.

FIG. 4 is a view into the flexible end effector looking down the direction of the shaft axis.

FIG. 5 is a view into an alternate flexible end effector looking down the direction of the shaft axis.

FIG. 6 is a view into another alternate flexible end effector looking down the direction of the shaft axis.

FIGS. 7A and 7B show the multiple rigid sections of FIG. 3 having a rigid sheath exposed in FIG. 7A and withdrawn in FIG. 7B.

### DETAILED DESCRIPTION OF THE DRAWINGS, INCLUDING THE PREFERRED EMBODIMENT

In the following detailed description of the invention, reference is made to the accompanying drawings which form a part hereof, and in which are shown, by way of illustration, specific embodiments in which the invention may be practiced. It is to be understood that other embodiments may be used, and structural changes may be made without departing from the scope of the present invention.

The flexible end effector accesses a body cavity through or alongside an endoscope or guide tube, and protrudes during use beyond the distal tip of the scope or guide tube. The extreme distal tip of the effector is provided with features useful to the surgeon/endoscopist, for example, but not limited to, serrated or rat-tooth forceps, biopsy forceps, cutting and electrocautery via plasma or electrical current, or the active end of a laser fiber. Control or operational wires of the features may be guided through center or other appropriate region of the end effector. A laser fiber, for example, could be disposed at or near the neutral axis of the bending portion to minimize destructive bending forces and to minimize the force required to bend to a desired angle.

The flexible end effector maintains a flexible bending section highly resistant to buckling. Layers of solid materials, metals or polymers, are disposed on either side of the device axis to allow bending around a single axis. Each layer consists of two or more flat springs, elongated in the direction of the shaft axis. High stiffness in the perpendicular direction due to the elastic mechanics of metals and polymers, provides a precise, repeatable motion allowing for 2-way deflection (I.e up/down or left/right, etc.) controlled by the physician or operator of the device control handle.

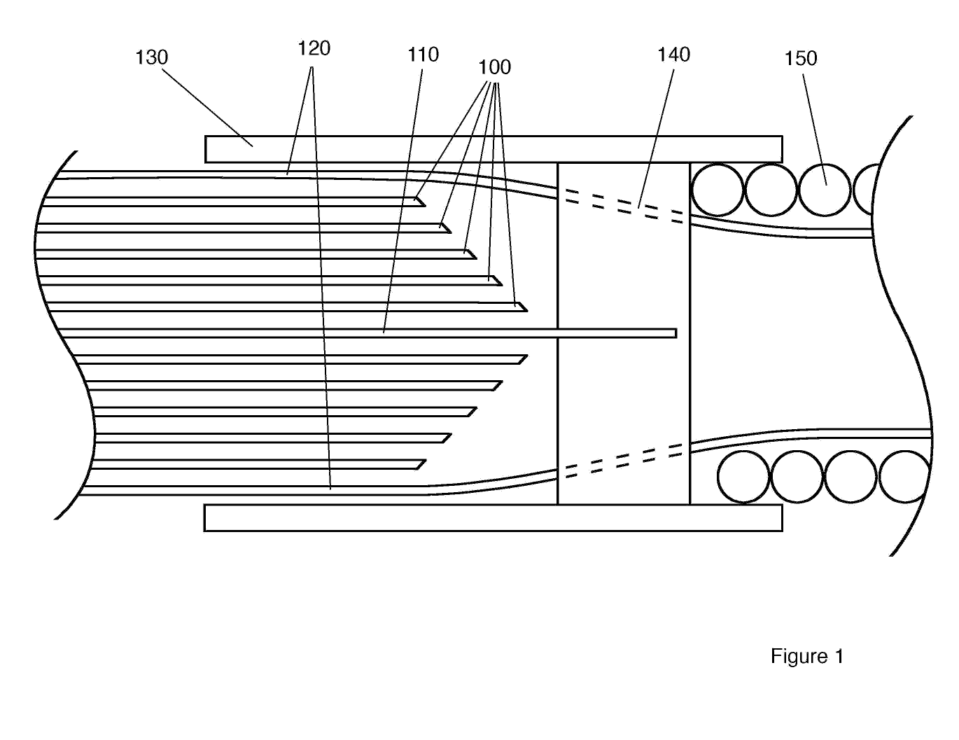

FIG. 1 shows a side or length-wise view of the flexible end effector connected to shaft 150. Flat spring layers 100 are spread on either side of central layer 110. Actuating layers 120 are positioned on the outer edge of the flexible end effector, connected to the distal end of the flexible section of the end effector, and cause deflection when compression is applied through anchor or control wire or connection 140. Ferrule or constraint 130 may surround the base of the flexible end effector to maintain orientation and connections.

The springs are preferably connected to one another at one end of the bending section. This allows sliding between layers during flexing, while maintaining the integrity of the device and precision of motion. Sliding friction may be reduced by various methods, including but not limited to interleaving materials with a low coefficient of friction such as floropolymers or polyethylene, applying low friction coatings, or by provision of solid, semi-solid, or liquid lubricants. Alternatively or additionally the spring layers may be encased in a polymeric or elastomeric sleeve. Alternative to the sleeves, and encapsulant, such as with a polymeric or elastomeric foam, may adhere to and around the spring layers, holding them substantially in place while allowing motion due to highly flexible properties of the foam.

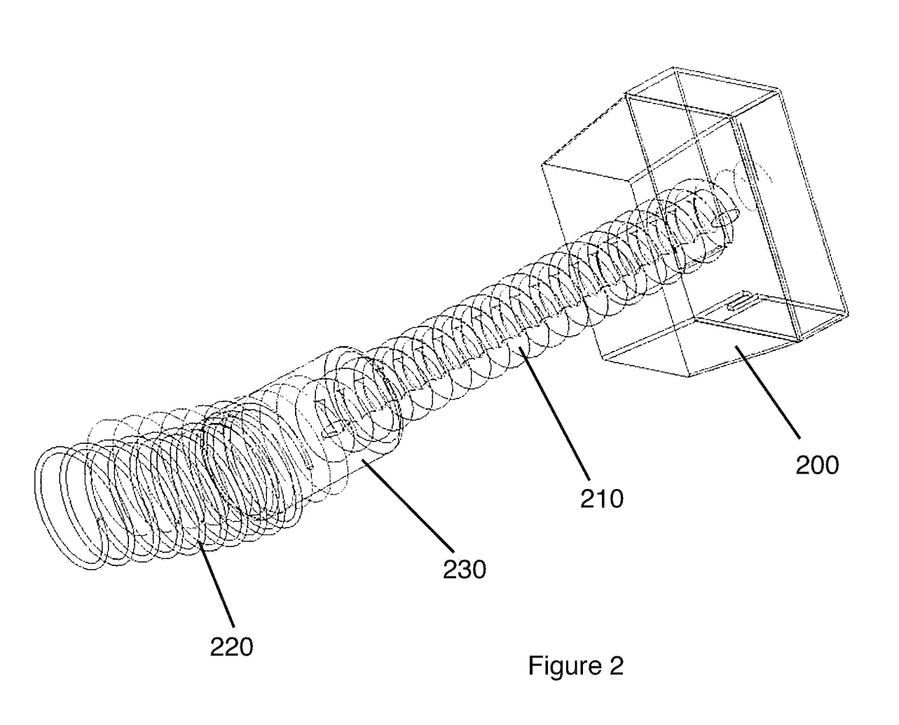

FIG. 2 shows the flexible section of the end effector connected with the entire device. Handle or control box 200 connects with shaft 210, which connects with the flexible end effector shown as constraining wire 220. Different means of connection between shaft and end effector may be implemented, including through ferrule 230 as shown.

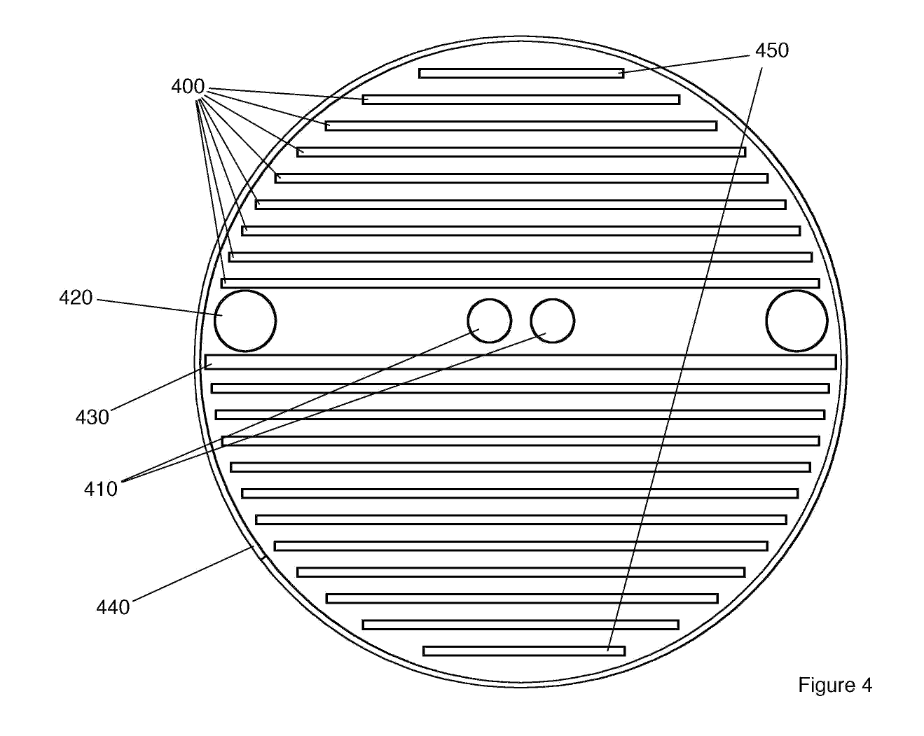

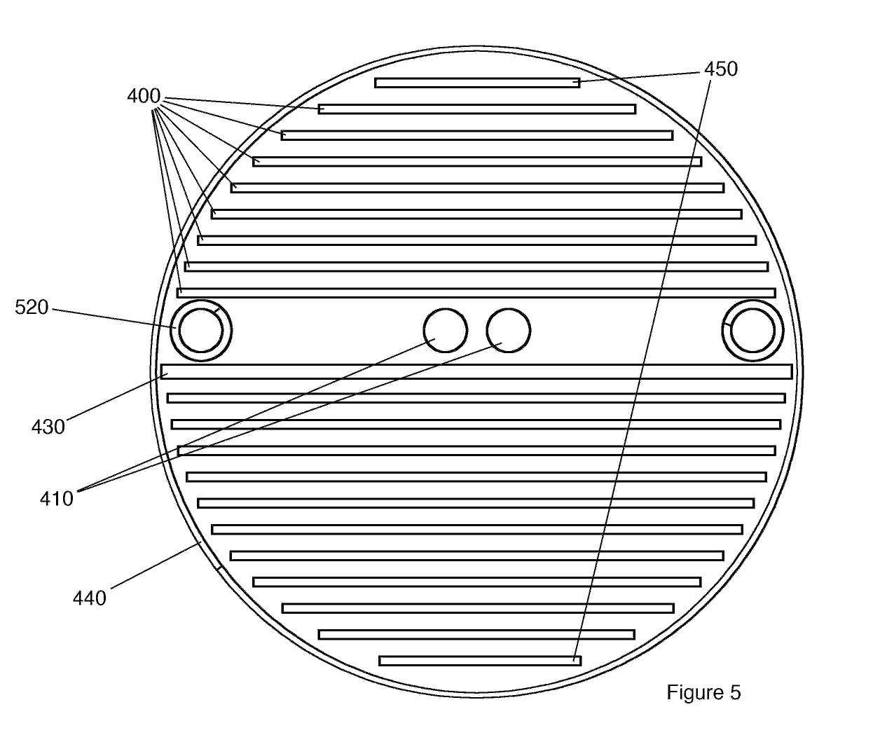

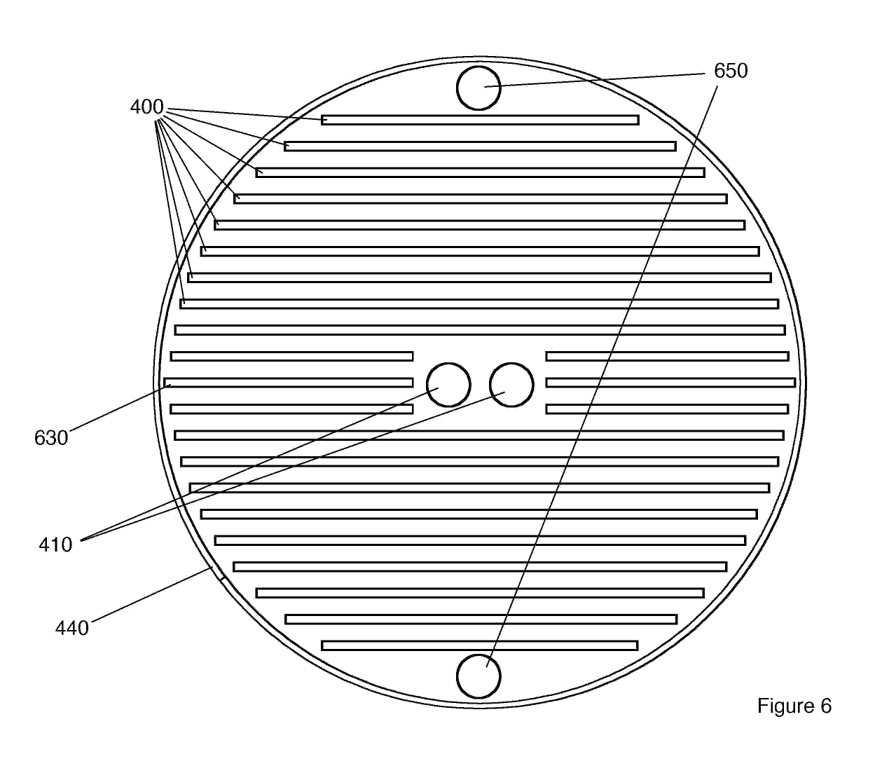

FIG. 4 shows a view into a flexible end effector. Flat springs 400 are arranged from the narrowest spring layer towards the edge to widest spring layer towards the center. Medical device or operational control wires 410 may be run through the center of the device. Spacers 420 may be round wires to maintain the central space and protect device or control wires 410 from pinching during operation. Center layer 430 may be thicker than flat springs 400 to absorb compressive load and prevent buckling. Actuating layers 450 receive tensile forces from controls running through the shaft to the handle, and cause the actual deflection of the flexible end effector. Constraining wire 440 may surround the base or entire flexible end effector to protect and maintain orientation of the device. FIG. 5 shows an alternate embodiment where spacer wires are replaced by extension or coil springs 520 which maintain the spacing with less rigidity. FIG. 6 shows another alternate embodiment with center layer 630 split to allow control or operational device wires 410 to run through the true center of the flexible end effector. Actuator layers 450 may be flat layers as shown in FIGS. 4 and 5, solid or multi-strand wires 650 as shown in FIG. 6, or alternate controls to steer the distal end of the flexible section of the end effector.

Depending on the elastic moduli of the materials chosen for the layers, and upon their number, shape, alignment, and length of each spring layer, a wide range of angulations is possible, from flexing only a few degrees to 180 degrees (a U bend) or beyond. Most typical applications are served by bending to an angle between 15 and 90 degrees. Two-way deflection (such as up/down) is sufficient for most purposes. Four-way deflection (such as both up/down and right/left) may be achieved by two adjacent, successive sections rotated 90 degrees to create perpendicular planes of deflection. Alternative 4-way deflection axis may be achieved by varying the degree of rotation between the adjacent sections. Due to the highly directional flexibility of the layered springs, each individual deflection section is limited to 2-way deflection.

The flexible end effector may be steered (deflected to one side or the other) by handle operating any means known to those familiar with the art, including individual wires or stranded cables of any material (for example, but not limited to, stainless steel, nitinol, or MP35N) or shape (such as, but not limited to, triangular, elliptical, oval, square, round, or rectangular in any aspect ratio) placed in tension and/or compression, electric motors, shape memory force generators, and pressure exerted via fluids. In order to keep the line of action of the wire/cable (or other actuating mechanism) aligned with the perimeter of the device, a fine wire compression spring can be provided at the periphery of the device.

All of the parts may be produced by various manufacturing practices. The spring leaves may be produced by shearing, stamping, machining, or photochemical etching. Springs may be manufactured with integral tabs for connection and alignment, or manufactured already joined as appropriate. For example, photochemical etching may accurately produce a large number of springs simultaneously. These may be produced in a pattern allowing folding into the desired shape while retaining attachment at one end. The folding may be controlled by features incorporated at the time of etching, such as areas etched to half thickness of the stock, or arranging rows of tiny holes (for example, 0.25 mm diameter), weakening the material in the desired location to allow for folding without compromising the integrity of the piece. The etching or holes allowing folding may be shaped or aligned to also define tabs, or fasteners, such as producing a threaded, notched, or barbed edge once folded. Fasteners in turn may be used to hold the springs into position and alignment by use of ferrules or other appropriate connectors. By utilizing mass production, the flexible end effectors may be produced at a manufactured cost allowing sanitary disposal of the device after a single use. Manufacture for reusable applications is also possible.

### Other Embodiments

An alternate embodiment may be optimized for use through a surgical trocar port passed through the skin and muscle tissue rather than an endoscope. This embodiment has a rigid tube with a closely fitting outer diameter, enabling airtight passage through the port, and a flexible end effector. The end effector enables steering of cutting tools, graspers, and other surgical implements. The steering itself may be the primary tool, such as in a device for retraction of the liver. Unlike typical hinged instruments, the flexible section in the end effector has a broad, gradual curve to allow a greater variety of access angles. The radius of the curve may be fixed or varied depending on the specific procedure or use required. Curve variation may be by preselection prior to insertion into the body cavity, or by a separate control that adjusts the radius during use.

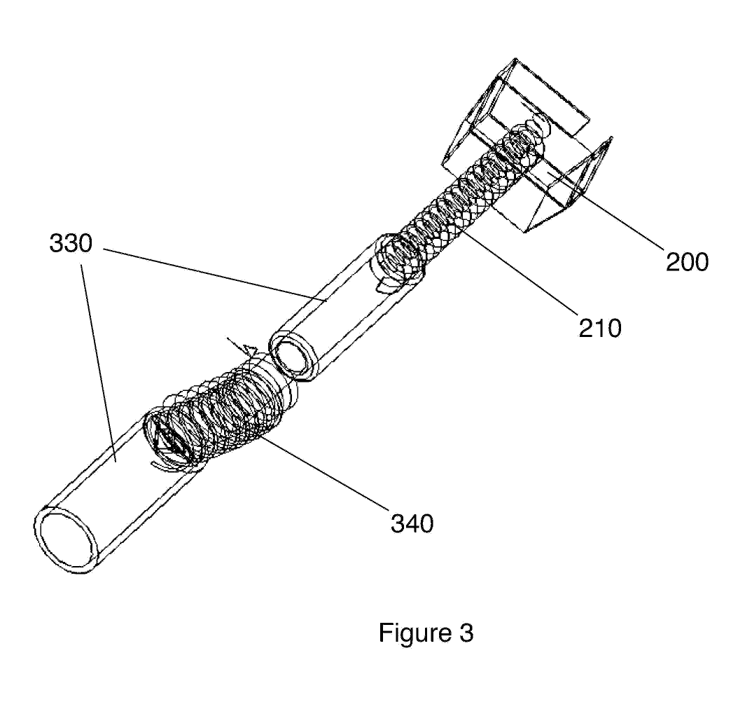

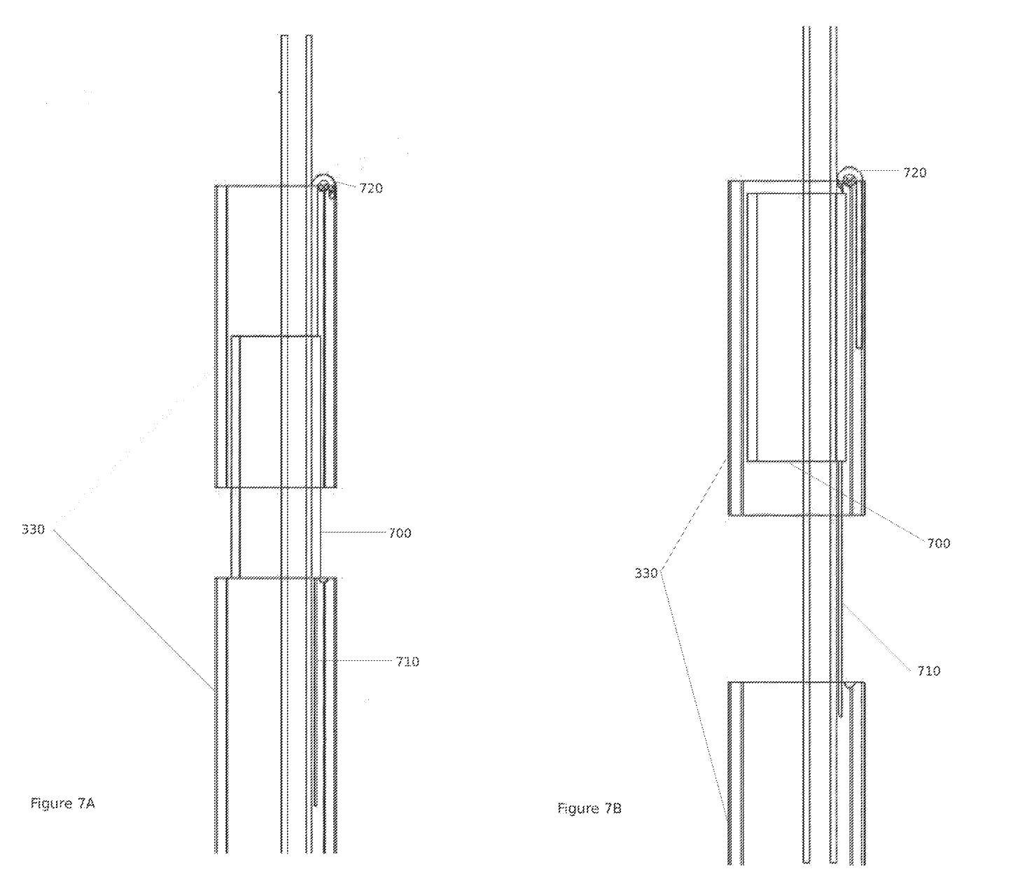

Another alternate embodiment uses multiple flexible sections located in the shaft in addition to the end effector to optimize for specific surgical procedures. For example, a retractor for bariatric surgery may have a flexible section with the distal end effector, and an additional flexible bending section between two or more longer rigid sections of the shaft, to allow positioning of the end effector around an intervening body structure, or to facilitate the triangulation and multiplication of forces. This embodiment is also useful in natural orifice surgery, when the point of entry is distant from the surgical site, as in a cholecystectomy using a vaginal entry and exit point. The flexible sections alternate with rigid sections in a pattern dictated by the particular procedure to be performed, or even in accordance with the individualities of the patients size and anatomy. A further variation on this embodiment is to provide for a means of making one or more of the flexible sections only temporarily active, by, for example, providing a rigid sheath that can be slid over the flexible section to render it inactive for part of the procedure. For example, a flexible portion may be “active” during insertion of the device, but may be rendered inactive during the cutting/grasping portion of the procedure. When rigidity is again needed at the end of the procedure, the stiffening tube is slid into position by wires or cables, and the device is withdrawn along a direct path. Alternatively, the flexibility can be activated and deactivated in such an order as allows insertion and withdrawal in an atraumatic manner. FIG. 3 shows one such alternate embodiment, with control handle 200 connected to shaft 210 having multiple rigid regions 330 connected by flexible sections within constraining wire 340. FIGS. 7A and 7B show that rigid sheath 700 may be exposed as in FIG. 7A or withdrawn as in FIG. 7B. When exposed, any flexible section covered by rigid sheath to prevent flexing. Sheath 700 may be held exposed such as by direct tension wire 710. Sheath 700 may be withdrawn by pulling an alternate wire or cable, such as over pulley 720. When withdrawn, any flexible section between rigid regions 330 may then be flexed. The tension/tension drive shown avoids the possibility of buckling and saves substantial space compared to traditional telescoping arrangements, where the longest tube extends through the entire length of the structure. It is to be understood that the above description is intended to be illustrative, and not restrictive.

Many other embodiments will be apparent to those of skill in the art upon reviewing the above description. The scope of the invention should, therefore, be determined with reference to the appended claims, along with the full scope of equivalents to which such claims are entitled.

What is claimed is:

1. A medical device comprising:
a handle;
a shaft connected to the handle; and
a flexible end effector connected to the shaft at an end opposite the handle, the flexible end effector comprising:
one or more layers disposed on either side of a device axis, each layer comprised of two or more flat springs elongated in the direction of the shaft axis;
a central region between the centermost layers for passage of control wires or operational elements of the medical device;
a connection to the handle at the exterior of end effector and aligned with the layers such that tightening the connection will flex the flexible end effector in the direction of the connection.

2. The medical device of claim 1, further comprising a spring connection between the layers of flat springs at one end of the flexible end effector.

3. The medical device of claim 2, further comprising interleaved layers between the layers of flat springs, the interleaved layers having a low coefficient of friction to allow sliding between flat spring layers during flex.

4. The medical device of claim 2, further comprising low friction coatings on the each flat spring layer.

5. The medical device of claim 2, wherein the flat spring layers are ordered in descending length from longest layers towards the center to shortest layers towards the exterior of the flexible end effector.

6. The medical device of claim 2, comprising an additional flexible region comprised of layers of flat springs elongated in the direction of the shaft axis, the additional flexible region positioned between the shaft and the flexible end effector and rotated from the orientation of the flexible end effector thereby allowing flex along a second axis to the flex of the flexible end effector.

7. The medical device of claim 2, wherein the flat springs layers contain fasteners on their side edges near the connection to the other layers such that the springs are connected to the flexible end effector by the fasteners to maintain spacing and orientation of the layers.
